(19) United States
(12) Patent Application Publication
(10) Pub. No.: US 2012/0165798 A1
(43) Pub. Date: Jun. 28, 2012

(54) DEVICE FOR TREATING EYE TISSUE

(76) Inventor: Christian Rathjen, Bremen (DE)

(21) Appl. No.: 13/324,051

(22) Filed: Dec. 13, 2011

Related U.S. Application Data
(60) Provisional application No. 61/426,869, filed on Dec. 23, 2010.

(51) Int. Cl. A61F 9/008

(52) U.S. Cl. 606/4

(57) ABSTRACT

An ophthalmological device for treating eye tissue, with a femtosecond laser oscillator for generating femtosecond laser pulses and with a light projector for projecting the laser pulses onto or into the eye tissue in a focused fashion is moreover provided with a picosecond laser module for generating picosecond laser pulses. In the process, femtosecond laser pulses and/or picosecond laser pulses can selectively be fed to the light projector for treating the eye tissue. Hence, the ophthalmological device can selectively be used for performing precise cuts in the eye tissue by means of the femtosecond laser pulses and for fragmenting eye tissue by tissue fragmentation by means of the picosecond laser pulses.

### CROSS-REFERENCE TO RELATED APPLICATIONS

The present application claims benefit of and priority to U.S. Provisional Patent Application Ser. No. 61/426,869 filed Dec. 23, 2010 entitled Vorrichtung zur Bearbeitung von Augengewebe, the entire contents of which are hereby incorporated by reference herein.

### BACKGROUND

1. Field of the Disclosure

The present invention relates to an ophthalmological device for treating eye tissue. The present invention more particularly relates to an ophthalmological device for treating eye tissue, including a femtosecond laser oscillator for generating femtosecond laser pulses and a light projector for projecting the laser pulses onto or into the eye tissue in a focused fashion.

2. Related Art

For decades laser technology has been publicized for universal use for processing the most diverse materials. In the field of ophthalmology, lasers are used for treating eye tissue, in particular for ablating, cutting and fragmenting eye tissue. For precise cuts in the eye tissue, in particular the cornea, for example for cutting a corneal flap within the scope of laser-assisted in situ keratomileusis (LASIK) for refractive correction of an eye, use is preferably made of very focused femtosecond laser pulses that have pulse widths (i.e. a temporal pulse length or pulse duration) of typically 100 fs to 1000 fs (1 fs=10⁻¹⁵ s). A femto laser system for cutting corneal flaps is described in e.g. EP 1 731 120. The femtosecond laser pulses are generated in a femtosecond beam source with a femtosecond laser oscillator, which generally operates with pulse rates in the MHz range. Femtosecond beam sources which are embodied as pure laser oscillators without downstream optical amplifiers are more stable, compact and cost-effective compared to femtosecond beam sources in a so-called MOPA (master oscillator power amplifier) configuration with a downstream optical amplifier. Femtosecond laser oscillators without downstream optical amplifiers however generate femtosecond laser pulses with significantly lower pulse energy than femtosecond beam sources in MOPA configuration. The limited pulse energy of a pure femtosecond laser oscillator, for example does not suffice to fragment eye tissue, for example, the tissue in a lens of the eye, by means of tissue fragmentation.

### SUMMARY

An object of the present invention is to propose an ophthalmological device, for treating eye tissue, that does not have at least some of the disadvantages of the known systems. In particular, an object of the present invention is to propose an ophthalmological device for treating eye tissue, including a femtosecond laser oscillator, which device is suitable both for performing precise, tissue-tolerated cuts in the eye tissue and for fragmenting eye tissue.

According to the present invention, these objects are achieved by the features of the independent claims. Further advantageous embodiments moreover emerge from the dependent claims and from the description.

The present invention achieves the aforementioned goals in particular by virtue of the fact that an ophthalmological device for treating eye tissue, including a laser pulse generator for generating laser pulses with a femtosecond laser oscillator for generating femtosecond laser pulses, and a light projector for projecting the laser pulses onto or into the eye tissue in a focused fashion, moreover including a picosecond laser module for generating picosecond laser pulses, wherein femtosecond laser pulses and/or picosecond laser pulses can selectively be fed to the light projector for treating the eye tissue.

The femtosecond laser pulses can be used to perform precise cuts in the eye tissue, for example in the cornea; here, the femtosecond laser pulses are projected into the tissue in a focused fashion with a pulse energy of between 10 nJ and 1000 nJ and a pulse rate in the region of 1-100 MHz, wherein substantially only the smallest amounts of the pulse energy propagate into the tissue adjoining the cut surface and hence there are no undesired adverse effects on the tissue. The picosecond laser pulses can be used to fragment the eye tissue by tissue fragmentation, for example in the lens of the eye; here, the picosecond laser pulses are projected into the tissue in a focused fashion with a comparatively higher pulse energy of between 1 μJ and 100 μJ and with a comparatively lower pulse rate in the region of 1 kHz-1 MHz. In contrast to femtosecond laser beam sources, picosecond laser beam sources are very robust and inexpensive at high and medium powers. The fact that, compared to femtosecond laser pulses, higher pulse energies are required for tissue interaction in the case of picosecond laser pulses, and that this is connected to significantly lower tissue tolerance, plays no role in applications such as fragmentation because the fragmented tissue is subsequently removed. Quite the opposite, the higher pulse energies are even desired because these can achieve a more effective tissue fragmentation.

In a preferred embodiment, the device includes a control module designed to selectively put the device into and control said device in at least one of the following operating modes:
a first operating mode, in which femtosecond laser pulses are fed to the light projector from the laser pulse generator for cutting the eye tissue,
a second operating mode, in which picosecond laser pulses are fed to the light projector from the laser pulse generator for fragmenting the eye tissue,
a third operating mode, in which, respectively with a temporally offset pulse start, a picosecond laser pulse followed by a femtosecond laser pulse are fed to the light projector from the laser pulse generator for treating the eye tissue, and
a fourth operating mode, in which, respectively with a temporally offset pulse start, a femtosecond laser pulse followed by a picosecond laser pulse are fed to the light projector from the laser pulse generator for treating the eye tissue.

By combining two successive and for example partly overlapping laser pulses in the third and fourth operating mode, the pulse energy of the two pulses can be suitably combined. This affords the possibility of utilizing novel interaction mechanisms which otherwise are inaccessible when only femtosecond or picosecond laser pulses are used. By way of example, a preceding femtosecond laser pulse can bring about initial igniting and the subsequent picosecond laser pulse can pump additional energy into the ignited interaction zone. Conversely, picosecond laser pulses below the optical penetration threshold can for example put the tissue into an activated state which allows lower pulse energies of the femtosecond laser pulses used for cutting; this in turn allows more precise cuts.

In one embodiment, the picosecond laser module includes a picosecond laser oscillator for generating the picosecond laser pulses.

In another embodiment, the picosecond laser module includes a pulse stretcher, which is designed to stretch femtosecond laser pulses from the femtosecond laser oscillator into picosecond laser pulses.

In a further embodiment, the picosecond laser module includes a detuning member, which can be switched on, is arranged in the femtosecond laser oscillator and is designed to modify the femtosecond laser oscillator in the switched-on state such that the femtosecond laser oscillator can be operated as a picosecond laser oscillator. In the switched-on state, the detuning member can for example restrict the spectral bandwidth of the femtosecond laser oscillator such that the femtosecond laser oscillator operates as a picosecond laser oscillator and generates picosecond laser pulses.

In one embodiment, provision is made for a polarization modulation such that the femtosecond laser pulses and the picosecond laser pulses have different polarizations, for example normally with respect to one another, and such that the device includes a polarization beamsplitter in order to selectively feed femtosecond laser pulses or picosecond laser pulses to the light projector. By way of example, a polarization modulator is downstream of the femtosecond laser oscillator and/or the picosecond laser module, or the femtosecond laser oscillator and/or the picosecond laser module are/is designed to polarize the femtosecond laser pulses and the picosecond laser pulses differently.

In a further embodiment, for selectively feeding femtosecond laser pulses or picosecond laser pulses to the light projector, provision is made for at least one controllable shutter, a controllable rotating mirror, an electromechanical switch and/or an electronic switch.

The femtosecond laser pulses can preferably be projected into the cornea in a focused fashion for cutting the eye tissue, and the picosecond laser pulses can preferably be projected into the lens of the eye in a focused fashion for fragmenting the eye tissue.

The laser pulse generator is preferably arranged in a common housing together with the femtosecond laser oscillator and the picosecond laser module, and the ophthalmological device includes a cooling device, connected to the housing, for thermalizing the laser pulse generator. This allows the femtosecond laser oscillator and the picosecond laser module to share the complicated and expensive cooling device for thermalization.

The device preferably includes a common electronic laser control module, connected to the femtosecond laser oscillator and the picosecond laser module, for controlling the femtosecond laser oscillator and the picosecond laser module, and/or a common feed unit, connected to the femtosecond laser oscillator and the picosecond laser module, for electrically feeding the femtosecond laser oscillator and the picosecond laser module. Thus, the femtosecond laser oscillator and the picosecond laser module can share the laser control module and/or the feed unit.

In one embodiment, a pulse selector for generating a specific pulse rate is downstream of the femtosecond laser oscillator, and the pulse rate can, for example, be set depending on a selected operating mode.

In a further exemplary embodiment, the device includes a common pumping source for the femtosecond laser oscillator and the picosecond laser module.

In one embodiment variant, the picosecond laser module includes an optical amplifier.

In a further embodiment variant, the picosecond laser module includes an optical compressor for compressing the pulse length of the picosecond laser pulses.

### BRIEF DESCRIPTION OF THE DRAWINGS

In the following text, an embodiment of the present invention is described on the basis of an example. The exemplary embodiment is illustrated by the following attached figures, in which:

FIG. 1 shows a block diagram that schematically illustrates an ophthalmological device with a laser pulse generator, which device selectively feeds femtosecond laser pulses and/or picosecond laser pulses, for focused projection, to a light projector for treating eye tissue,

FIG. 2 shows a block diagram that schematically illustrates an embodiment of the ophthalmological device, in which the laser pulse generator includes a femtosecond laser oscillator and a picosecond laser oscillator,

FIG. 3 shows a block diagram that schematically illustrates an embodiment of the ophthalmological device, in which femtosecond laser pulses and picosecond laser pulses are fed to the light projector via polarization modules and a polarizing beamsplitter,

FIG. 4 shows a block diagram that schematically illustrates an embodiment of the ophthalmological device, in which the laser pulse generator includes a femtosecond laser oscillator and a pulse stretcher for generating the picosecond laser pulses,

FIG. 5 shows a block diagram that schematically illustrates an embodiment of the ophthalmological device, in which the laser pulse generator includes a femtosecond laser oscillator and a detuning member arranged therein for generating the picosecond laser pulses, and

FIGS. 6a-6d schematically illustrate, from top to bottom, a femtosecond laser pulse, stretching of the femtosecond laser pulse, optical amplification of the stretched pulse and a compression of the amplified and stretched pulse.

### DETAILED DESCRIPTION OF THE EMBODIMENTS

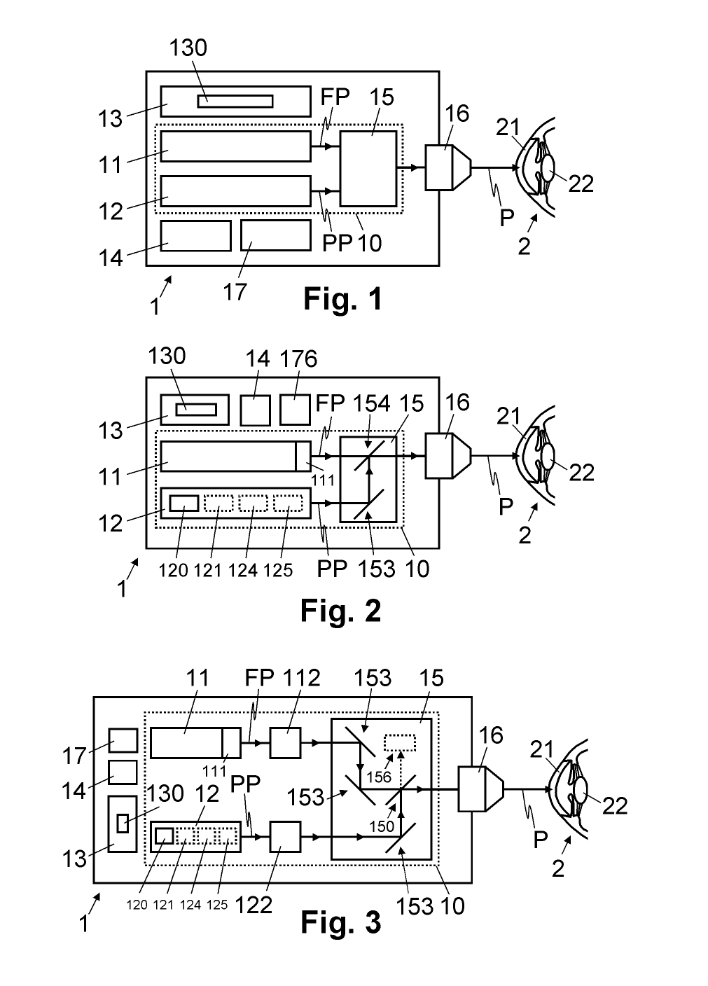

In FIGS. 1 to 5, reference sign 1 relates to an ophthalmological device for treating tissue in an eye 2, more particularly eye tissue of the cornea 21 and the lens of the eye 22. The ophthalmological device 1 includes a laser pulse generator 10 for generating laser pulses P and a light projector 16 for projecting the laser pulses P onto or into the eye tissue in a focused fashion, more particularly onto or into the cornea 21 and/or the lens of the eye 22.

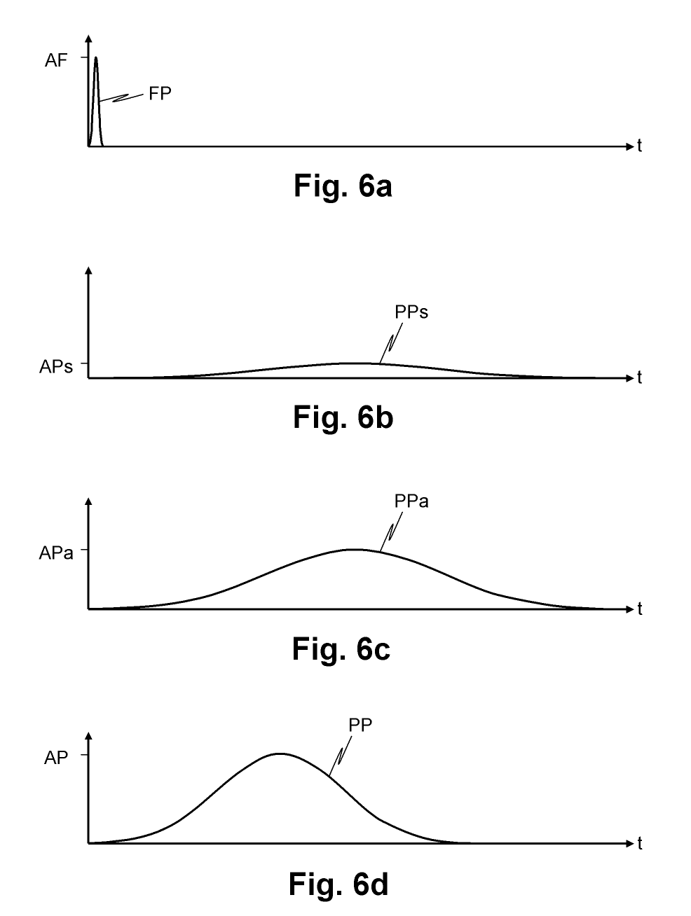

As illustrated schematically in FIGS. 1 to 5, the laser pulse generator 10 includes a femtosecond laser oscillator 11 for generating femtosecond laser pulses FP. FIG. 6a schematically illustrates the time profile of a femtosecond laser pulse FP with an amplitude AF. A pulse selector 111 is preferably, but not necessarily, downstream of the femtosecond laser oscillator 11 and designed to transmit the femtosecond laser pulses FP at a specific pulse rate.

The laser pulse generator 10 moreover includes a picosecond laser module 12 for generating picosecond laser pulses PP. Picosecond laser pulses PP have a pulse width (temporal pulse length or pulse duration) of typically 5 ps to 1000 ps (1 ps=10⁻¹² s). In the following text, various variants for generating the picosecond laser pulses PP are described with reference to FIGS. 2 to 5.

In the variants as per FIGS. 2 and 3, the picosecond laser module 12 includes a picosecond laser oscillator 120 for generating the picosecond laser pulses PP. The ophthalmological device 1 preferably includes a common pumping source for the femtosecond laser oscillator 11 and the picosecond laser module 12. Depending on the embodiment, a pulse selector 121 is moreover downstream of the picosecond laser oscillator 120 and designed to transmit the picosecond laser pulses PP at a specific pulse rate. Moreover, as illustrated schematically in FIGS. 2 and 3, an optical amplifier 124 for amplifying the picosecond laser pulses PP and/or a compressor 125 for compressing the pulse length of the picosecond laser pulses PP is/are optionally downstream of the picosecond laser oscillator 120.

A person skilled in the art will understand that the amplifier of the picosecond laser module 12 can also be designed in the form of a regenerative amplifier, a multipass amplifier or as a multistage amplifier.

A person skilled in the art will moreover understand that fibers and/or crystals can be used as active media in the laser oscillators and amplifiers.

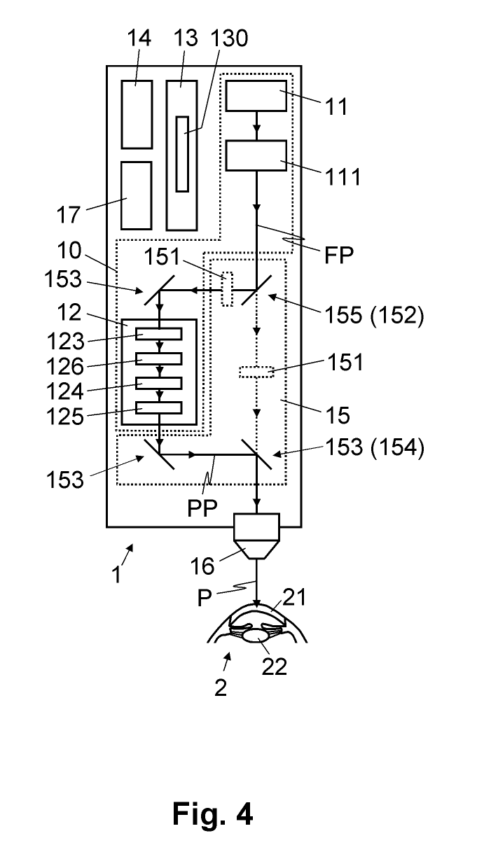

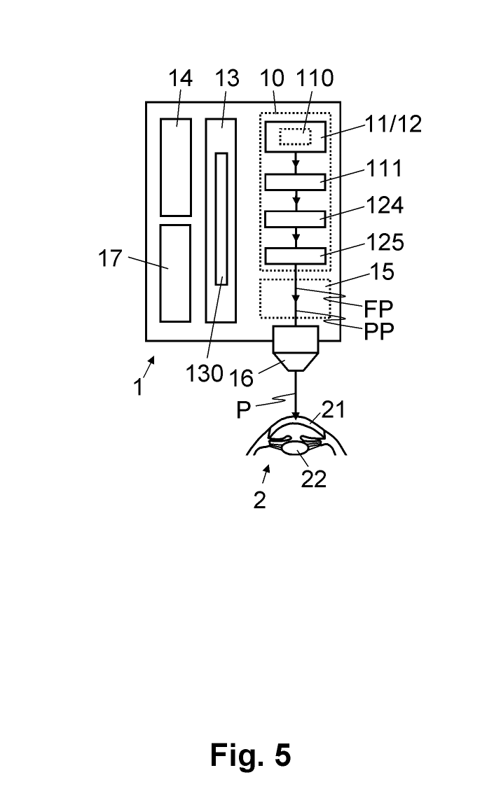

In the variants as per FIGS. 4 and 5, the picosecond laser module 12 includes a pulse converter, which is designed to generate the picosecond laser pulses PP from the femtosecond laser pulses FP generated by the femtosecond laser oscillator 11.

In the variant as per FIG. 4, the pulse converter is embodied in the form of a pulse stretcher 123 arranged in the picosecond laser module 12. The pulse stretcher 123 stretches the femtosecond laser pulses FP to a pulse width or pulse duration of a picosecond laser pulse PP. FIG. 6b schematically illustrates a picosecond laser pulse PPs emerging from the femtosecond laser pulse FP illustrated in FIG. 6A being stretched, wherein the amplitude AF of the femtosecond laser pulse FP is attenuated during the stretching to the amplitude APs of the picosecond laser pulse PPs. As illustrated schematically in FIG. 4, an optical amplifier 124 for amplifying the picosecond laser pulses PP is optionally downstream of the pulse stretcher. FIG. 6c schematically illustrates an amplified picosecond laser pulse PPa with the increased amplitude APa, which emerges from the optical amplification of the stretched pulse PPs illustrated in FIG. 6b. A person skilled in the art will understand that, depending on the power thereof, femtosecond laser pulses FP are attenuated in an optical attenuation module before being fed to the optical amplifier 124 of the picosecond laser module in order to avoid overloading the optical amplifier 124. In one embodiment, the femtosecond laser pulses FP are stretched excessively, i.e. longer than the defined pulse length of the picosecond laser pulses PP to be emitted, as a result of which the femtosecond laser pulses FP are attenuated such that an optical attenuation module can be dispensed with. The picosecond laser module 12 then accordingly includes an optical compressor 125 for compressing the pulse length of the excessively stretched femtosecond laser pulses PPs to the defined pulse length of the picosecond laser pulses PP to be emitted. FIG. 6d schematically illustrates a picosecond laser pulse PP with a further amplified amplitude AP, emerging from the compression of the excessively stretched picosecond laser pulse PPs illustrated in FIG. 6c.

In the variant as per FIG. 5, the pulse converter is embodied in the form of a detuning member 110, which can be switched on and is arranged in the femtosecond laser oscillator 11. The detuning member 110 is designed to modify the femtosecond laser oscillator 11 in the switched-on state such that the femtosecond laser oscillator 11 operates as a picosecond laser oscillator 12 and generates picosecond laser pulses PP. By way of example, the detuning member 110 restricts the spectral bandwidth of the femtosecond laser oscillator 11 such that the latter is operated as a picosecond laser oscillator 12 in the switched-on state of the detuning member 110. By way of example, the detuning member 110 is embodied as a spectral filter. As illustrated schematically in FIG. 5, a pulse selector 111 for determining the pulse rate, an optical amplifier 124 for amplifying the femtosecond laser pulses FP or picosecond laser pulses PP and/or a compressor 125 for compressing the pulse length of the femtosecond laser pulses FP or picosecond laser pulses PP are/is optionally downstream of the femtosecond laser oscillator 11.

The reference sign 15 relates to an optical transmission system for feeding the laser pulses generated by the laser pulse generator 10 to the light projector 16. Depending on the embodiment, the optical transmission system 15 includes polarization beamsplitters 150, shutters 151, beamsplitters 152, fixed (deflection) mirrors 153, beam-unifying elements 154, e.g. partly transmissive mirrors, controllable rotating mirrors 155 and/or optical waveguides. By way of example, the shutters 151 are respectively embodied with a controllable rotating mirror in combination with an optical beam-dump module. In the transmissive, interconnected state, the laser pulses are fed to the desired load by the rotating mirror of the shutter 151, for example to the light projector 16 for focused projection into or onto the eye tissue. By contrast, in the non-transmissive, blocked state, the laser pulses are fed to the beam-dump module by the rotating mirror of the shutter 151, where they are absorbed. In further variants, the shutter is embodied in an electro-optical or mechanical fashion, for example as a stop.

In FIGS. 1 to 5, reference sign 17 relates to a common feed unit for electrically feeding the femtosecond laser oscillator 11 and the picosecond laser module 12.

The femtosecond laser oscillator 11 and the picosecond laser module 12 of the laser pulse generator 10 are arranged in a common housing. In order to thermalize the laser pulse generator 10, i.e. the femtosecond laser oscillator 11 and the picosecond laser module 12, the ophthalmological device 1 includes a cooling device 14 connected to this housing. That is to say the femtosecond laser oscillator 11 and the picosecond laser module 12 are thermalized by a common cooling device 14.

Furthermore, if a common housing is utilized it is possible to make common use of optical elements (pulse selectors, e.g. mirrors), depending on the design of the individual beam sources.

The selective feed of the femtosecond laser pulses FP and/or picosecond laser pulses PP to the light projector 16 has different embodiments in various embodiment variants. In some variants, the selective feed of the femtosecond laser pulses FP and/or picosecond laser pulses PP to the light projector 16 is brought about by the optical transmission system 15.

In the embodiment as per FIG. 2, the selection of femtosecond laser pulses FP or picosecond laser pulses PP is made by corresponding switching on and off of the laser oscillators, i.e. by switching on the femtosecond laser oscillator 11 when the picosecond laser oscillator 120 is switched off or by switching on the picosecond laser oscillator 120 when the femtosecond laser oscillator 11 is switched off. Controllable electromechanical switches and/or electronic switches are provided for switching the laser oscillators on and off. In one variant, the aforementioned pulse selectors are used as switches. In order to switch between femtosecond laser oscillator 11 and picosecond laser oscillator 120, shutters 151 for suppressing laser pulses can moreover also be downstream of the laser oscillators. In order to be fed to the light projector 16, the femtosecond laser pulses FP and picosecond laser pulses PP are routed to a common projection axis via deflection mirrors 153 and beam-unifying elements 154, e.g. partly transmissive mirrors.

In the embodiment as per FIG. 3, the selection of femtosecond laser pulses FP or picosecond laser pulses PP is made on the basis of a different polarization. It can be seen in FIG. 3 that there respectively is one polarization modulator 112, 122 downstream of the femtosecond laser oscillator 11 and/or the picosecond laser oscillator 120, which polarization modulators transmit the femtosecond laser pulses FP and picosecond laser pulses PP with different polarizations, for example with a mutually normal polarization. To this end, the polarization modulators 112, 122 are designed to rotate the polarization of the laser pulses to a different extent or to polarize the laser pulses in a different fashion. The differently polarized femtosecond laser pulses FP and picosecond laser pulses PP are fed to a controllable polarization beamsplitter 150 via deflection mirrors 153, which polarization beamsplitter, depending on how it is being actuated and on the polarization of the laser pulses, selectively transmits the femtosecond laser pulses FP or the picosecond laser pulses PP to the light projector 16 for projection onto or into the eye tissue. Non-transmitted laser pulses are for example fed to an optional beam-dump module 156. In a simplified and preferred embodiment, the femtosecond laser oscillator 11 and/or the picosecond laser oscillator 120 are/is designed to emit the femtosecond laser pulses FP and picosecond laser pulses PP with different polarizations, and so downstream polarization modulators 112, 122 can be dispensed with.

In the variant as per FIG. 4, the selection of femtosecond laser pulses FP or picosecond laser pulses PP is made by means of a controllable rotating mirror 155 or by means of a combination of beamsplitter 152 and shutters 151. In this variant, the femtosecond laser pulses FP are selectively fed directly to the light projector 16 by the rotating mirror 155 or they are routed to the light projector 16 through the pulse stretcher 123 arranged in the picosecond laser module 12 via further deflection mirrors 153 in order to generate picosecond laser pulses PP. In the other variant, in each case via a controllable shutter 151, the beamsplitter 152 either feeds the femtosecond laser pulses FP to the light projector 16 along the direct path or routes said femtosecond laser pulses to the light projector 16 through the pulse stretcher 123 arranged in the picosecond laser module 12, the optional optical amplifier 124 and the optional compressor 125 via deflection mirrors 153. Hence femtosecond laser pulses FP and/or picosecond laser pulses PP can be fed to the light projector 16 by appropriate opening or closing of the shutters 151.

In the variant as per FIG. 5, the selection of femtosecond laser pulses FP or picosecond laser pulses PP is made by appropriate connection or disconnection of the detuning member 110.

In FIGS. 1 to 5, the reference sign 13 relates to an electronic control module for controlling the ophthalmological device 1. The control module 13 is preferably embodied as a programmed software module that controls one or more processors in the ophthalmological device 1 for executing functions described below and is stored in a fixed or removable, accessible, e.g. non-transient, computer-readable storage medium connected to the processors. A person skilled in the art will understand that the control module, or at least some of the functions thereof, can be partly or wholly embodied using hardware-based components in alternative embodiment variants.

Depending on user instructions, the control module 13 puts the ophthalmological device 1 into one of a number of operating modes described below and controls the components and functional modules of the ophthalmological device 1 according to the set operating mode.

In a first operating mode, for cutting the eye tissue, the femtosecond laser pulses FP are fed to the light projector 16 for the focused projection into the eye tissue, more particularly into the cornea 21. Depending on the embodiment variant, the femtosecond laser oscillator 11 is for this purpose switched on while the picosecond laser oscillator 120 is switched off; the polarization beamsplitter 150 is set for transmitting the polarization of the femtosecond laser pulses FP and for not transmitting the polarization of the picosecond laser pulses PP and optionally, depending on the embodiment variant, the polarization of the femtosecond laser pulses FP is modified for transmission and the polarization of the picosecond laser pulses PP is modified for non-transmission by the polarization beamsplitter 150; the femtosecond laser pulses FP are fed directly to the light projector 16 by the rotating mirror 155 or the beamsplitter 152 and shutters 151; or the detuning member 110 in the femtosecond laser oscillator 11 is switched off or disconnected.

In a second operating mode, for fragmenting the eye tissue, the picosecond laser pulses PP are fed to the light projector 16 for the focused projection into the eye tissue, more particularly into the lens of the eye 22. Depending on the embodiment, the picosecond laser oscillator 12 is for this purpose switched on while the femtosecond laser oscillator 11 is switched off; the polarization beamsplitter 150 is set for transmitting the polarization of the picosecond laser pulses PP and for not transmitting the polarization of the femtosecond laser pulses FP and optionally, depending on the embodiment variant, the polarization of the picosecond laser pulses PP is modified for transmission and the polarization of the femtosecond laser pulses FP is modified for non-transmission by the polarization beamsplitter 150; the femtosecond laser pulses FP are routed to the light projector 16, through the pulse stretcher 123 arranged in the picosecond laser module 12 for generating picosecond laser pulses PP, by the rotating mirror 155 or the beamsplitter 152 and shutters 151; or the detuning member 110 in the femtosecond laser oscillator 11 is switched on or connected for generating picosecond laser pulses PP.

In a third and a fourth operating mode, the picosecond laser pulses PP and femtosecond laser pulses FP are fed in combination to the light projector 16 for treating the eye tissue. Here, a sequence of laser pulses respectively combined in pairs is fed to the light projector 16. In the combined feed of laser pulses, respectively one picosecond laser pulse PP and one femtosecond laser pulse FP, either a femtosecond laser pulse FP followed by a picosecond laser pulse PP or a picosecond laser pulse PP followed by a femtosecond laser pulse FP, are fed with a temporally offset pulse start to the light projector 16 for the focused projection at the same work point. In one variant, the directly successive laser pulses in a pulse combination overlap in time. In order to generate a laser pulse combination, depending on the embodiment variant, the picosecond laser oscillator 120 and the femtosecond laser oscillator 11 are respectively operated with appropriate time-offset pulse generation, i.e. with appropriate phase shift between the start of the pulse, for example in the variants as per FIGS. 2 and 3, or in the case of synchronized pulse generation, in each case without a phase shift between the femtosecond laser pulse FP and picosecond laser pulse PP, said oscillators are embodied and operated with an appropriate interconnectable optical delay element 126, for example in the variants as per FIG. 4. The delay element 126 is preferably arranged in front of the optical amplifier 124 in the beam path, i.e. upstream of the optical amplifier 124.

By combining two successive and for example partly overlapping laser pulses in the third and fourth operating mode, the pulse energy and/or the pulse intensities of the two pulses can be suitably combined. By way of example, a preceding femtosecond laser pulse FP can bring about initial igniting and the subsequent picosecond laser pulse PP can pump additional energy into the ignited interaction zone. In the combined operation, the eye tissue can also be treated with nanosecond laser pulses in the upper nano-range, for example in the region of between 1 ns and 10 ns, in particular for fragmenting the lens. If the device 1 is designed to generate “clean” nanosecond laser pulses without spikes and/or amplitude variations, it can then also be used for cutting. In order to generate the nanosecond laser pulses, the femtosecond laser pulses FP or picosecond laser pulses PP are stretched accordingly or an appropriate detuning member 110 is connected. A person skilled in the art will moreover understand that the ophthalmological device 1 is moreover equipped with wavelength-converting elements in further embodiment variants.

The control module 13 is moreover designed to set the pulse rate depending on the selected operating mode and/or on the basis of user instructions, and to actuate the pulse selector 111 accordingly.

The control module 13 moreover includes a common electronic laser control module 130 for controlling the femtosecond laser oscillator 11 and the picosecond laser module 12, more particularly the picosecond laser oscillator 120. The laser control module 130 is designed to control the pulse energy, pulse width, maximum pulse intensity (depending on the pulse shape there are different values for the pulse intensity in the case of otherwise unchanging pulse width and pulse energy), pulse rate, wavelength, focal size and/or mean laser power of the laser oscillators.

Finally, it should be noted that the ophthalmological device 1 moreover includes beam-deflecting means (not illustrated) for local scanning of the eye tissue to be treated by means of the femtosecond laser pulses FP and/or picosecond laser pulses PP.

What is claimed is:

1. An ophthalmological device for treating eye tissue, comprising:
a laser pulse generator for generating laser pulses,
a light projector for projecting the laser pulses onto or into the eye tissue in a focused fashion,
the laser pulse generator including a femtosecond laser oscillator for generating femtosecond laser pulses, and
a picosecond laser module for generating picosecond laser pulses, wherein femtosecond laser pulses and/or picosecond laser pulses are selectively fed to the light projector for treating the eye tissue.

2. The device as claimed in claim 1, further comprising a control module configured to selectively put the device into and control said device in at least one of the following operating modes:
a first operating mode, in which femtosecond laser pulses are fed to the light projector from the laser pulse generator for cutting the eye tissue,
a second operating mode, in which picosecond laser pulses are fed to the light projector from the laser pulse generator for fragmenting the eye tissue,
a third operating mode, in which, respectively with a temporally offset pulse start, a picosecond laser pulse followed by a femtosecond laser pulse are fed to the light projector from the laser pulse generator for treating the eye tissue, and
a fourth operating mode, in which, respectively with a temporally offset pulse start, a femtosecond laser pulse followed by a picosecond laser pulse are fed to the light projector from the laser pulse generator for treating the eye tissue.

3. The device as claimed in claim 1, wherein the picosecond laser module comprises a picosecond laser oscillator for generating the picosecond laser pulses.

4. The device as claimed in claim 1, wherein the picosecond laser module comprises a pulse stretcher, which is configured to stretch femtosecond laser pulses from the femtosecond laser oscillator into picosecond laser pulses.

5. The device as claimed in claim 1, wherein the picosecond laser module comprises a detuning member, arranged in the femtosecond laser oscillator and configured to modify the femtosecond laser oscillator when switched-on such that the femtosecond laser oscillator is operable as a picosecond laser oscillator.

6. The device as claimed in claim 1, wherein the femtosecond laser pulses and the picosecond laser pulses have different polarizations and wherein the device comprises a polarization beamsplitter in order to selectively feed femtosecond laser pulses or picosecond laser pulses to the light projector.

7. The device as claimed in claim 1, wherein, the polarization beamsplitter selectively feeds femtosecond laser pulses or picosecond laser pulses to the light projector, using at least one of: a controllable shutter, a controllable rotating mirror, an electromechanical switch and an electronic switch.

8. The device as claimed in claim 1, wherein the femtosecond laser pulses are projected into the cornea in a focused fashion for cutting the eye tissue, and wherein the picosecond laser pulses are projected into the lens of the eye in a focused fashion for fragmenting the eye tissue.

9. The device as claimed in claim 1, wherein the laser pulse generator is arranged in a common housing together with the femtosecond laser oscillator and the picosecond laser module, and wherein the ophthalmological device further comprises a cooling device, connected to the housing, for thermalizing the laser pulse generator.

10. The device as claimed in claim 1, further comprising a common electronic laser control module, connected to the femtosecond laser oscillator and the picosecond laser module, for controlling the femtosecond laser oscillator and the picosecond laser module, and a common feed unit, connected to the femtosecond laser oscillator and the picosecond laser module, for electrically feeding the femtosecond laser oscillator and the picosecond laser module.

11. The device as claimed in claim 1, further comprising a pulse selector for generating a specific pulse rate positioned downstream of the femtosecond laser oscillator, and wherein the pulse rate is set depending on a selected operating mode.

12. The device as claimed in claim 1, further comprising a common pumping source for the femtosecond laser oscillator and the picosecond laser module.

13. The device as claimed in claim 1, wherein the picosecond laser module comprises an optical amplifier.

14. The device as claimed in claim 1, wherein the picosecond laser module comprises an optical compressor for compressing the pulse length of the picosecond laser pulses.

15. The device as claimed in claim 1, wherein the laser pulse generator is configured to generate femtosecond laser pulses with a pulse rate in the region of 1-100 MHz and picosecond laser pulses with a pulse rate in the region of 1 kHz-1 MHz.
